(19) United States
(12) Patent Application Publication
(10) Pub. No.: US 2009/0221949 A1
(43) Pub. Date: Sep. 3, 2009

(54) Device and method for circulatory isolation and treatment of a part of a body

(76) Inventors: Mats Allers, Lund (SE); Lars Eriksson, Horby (SE); Lars Holmberg, Nyland (SE); Frank Schanne, Kirkel (DE)

Correspondence Address: Schiff Hardin;Patent Department, 6600 Sears Tower, Chicago, IL, 60606-6473, US

(21) Appl. No.: 10/505,349

(22) PCT Filed: Feb. 21, 2003
(86) PCT No.: PCT/SE03/00284

§ 371 Date: Aug. 20, 2004

(30) Foreign Application Priority Data
Feb. 25, 2002 (SE) 0200579-1
Jul. 11, 2002 (SE) 0202172-3

(51) Int. Cl. A61M 1/36; A61M 25/00; A61B 17/00; A61M 25/10; A61M 1/00; A61F 7/00

(52) U.S. Cl. 604/6.16; 604/524; 606/201; 604/96.01; 604/151; 604/113; 604/9

(57) ABSTRACT

An apparatus for circulatory isolation and treatment of a part of a patient's body includes a fluid circulation loop having a first end and a second end, a flow obstructer adapted for obstructing blood flow in a patient's blood vessel such that a body part or organ becomes circulatorily isolated from the circulation of the rest of the body, and a flow-through arrangement including first and second flow-through members respectively connected to the first and second ends of the fluid circulation loop, for providing the isolated part of the patient's body with a fluid connection to the fluid circulation loop, allowing circulation of a therapeutic fluid through the isolated body part.

### BACKGROUND OF THE INVENTION

1. Field of the Invention

The present invention relates to the field of medical technology, specifically to devices and methods for circulatory isolation and treatment of a part of a body.

2. Description of the Prior Art

There is a need today for ways of treating conditions such as thrombosis and non-disseminated type cancer using efficient, fast, and cost-effective methods. There is a need for being able to use high concentrations of therapeutic agents for a prolonged time without causing side effect injuries to sensitive organs such as the brain, and without causing oxygen deficiency and breakdown product accumulation in the treated body part.

Thrombosis is a condition where a thrombus or blood clot is obstructing or blocking the normal flow of blood trough a vessel. Thrombosis in an artery in an extremity can lead to anoxia and may even require that the extremity be amputated.

Conventionally, treatment of thrombosis is medical or surgical. Known methods for medical treatment are relatively inexpensive but are mainly preventive types of treatment, involving oral drugs preventing thrombus build-up, and intravenous drugs both of the anti-thrombus build-up kind and the thrombolytic kind. Surgical treatment on the other hand, is mainly for treating acute thrombosis, and known surgical methods tend to be relatively expensive.

PCT Application WO 01/70325 to Kokish et al., discloses an emboli protection system that provides one or more inflatable blocking balloons, introducible into a blood vessel, for isolation of a section of said vessel to prevent the migration of emboli from the section during an interventional procedure, and fluid infusion and evacuation ports for flushing emboli from the isolated section. The blocking balloons can be perforated to provide the infusion ports, and thrombolytic inflation fluid may be used to break down and dissolve thrombus and plaque in the isolated portion of the blood vessel.

U.S. Pat. No. 4,540,399 to Litzie et al., discloses a closed emergency heart bypass system for extra-corporeal blood circulation using few components. The components include a non-occlusive blood pump aspirating venous blood from an appropriate cannula for introduction to an oxygenator and a bubble-trapping device followed by return to a patient's body via an arterial cannula. Tubing interconnects the components and a bypass loop selectively joins the tubing adjacent to the venous and arterial cannulas for air displacement during initial pump priming and tube purging.

In an article by Mumme (Fortschr. Med. 113. Jg (1995), Nr. 13 A. Mumme, Fibrinolytikaperfusion) a method for hyperthermal fibrinolytic perfusion in an isolated extremity is described where a heart-lung machine is used to provide circulatory energy.

### SUMMARY OF THE INVENTION

An object of the present invention is to respond to the above-mentioned need by providing an apparatus that is able to isolate a part of the blood circulatory system from the rest of the system and that is able to circulate a therapeutic fluid in said isolated part, while at the same time providing oxygen to that part.

The apparatus according to the invention includes the basic components of a fluid circulation loop having a first end and a second end, a flow obstructer adapted to obstruct blood flow in a patient's blood vessel, so that a body part or organ of the patient becomes circulatory isolated from the circulation of the rest of the patient's body, and a flow-through arrangement formed by a first flow-through member and a second flow-through member respective connectible to the first and second ends of the fluid circulation loop, for providing the isolated part of the patient's body with a fluid connection to the fluid circulation loop, allowing circulation of a therapeutic fluid through the isolated body part.

In a preferred embodiment the flow obstructer is a compressing element with a height (h), applicable around said extremity, devised to compress a section of said extremity and thereby circulatorily isolate the patient's extremity and prevent a leakage of a fluid, that can be blood or a therapeutic liquid, from said extremity to the rest of the patient's circulation, and the flow-through arrangement is formed by catheters, having a reinforced section with a length (L) greater than the height (h) of the compressing element, devised for being introduced in an artery and in a vein having connection to the extremity.

In another preferred embodiment flow obstructer and flow-through arrangement are balloon catheters being capable of obstructing the blood flow in a patient's blood vessels leading to and from an isolated part or organ of the body.

This present apparatus is useable for flushing the blood circulatory subsystem of an extremity by introducing the catheters into a main artery and vein, respectively, of the extremity, in a distal direction, such that compressing means can be applied around the extremity distal to the entry site of the catheters, but proximal to the free ends of the catheters such that fluid can be pumped through the arterial catheter, into the main arterial vessel of the extremity and such that the liquid can return via the main venous vessel of the extremity to the venous catheter and a venous side of the fluid circulation loop. As used herein free ends means those ends of the incompressible catheters that are devised to connect to the main artery respective vein of the extremity, i.e. that are not devised to be connected to the fluid circulation loop.

The compressing is designed, when applied on the outside of an extremity, to exert a minimum inward pressure that is sufficient to shut off the blood flow through the corresponding cross-section of the extremity, and wherein the catheters, arranged to pass through this cross-section, are devised to endure the pressure, making it possible to access the blood vessels normally supplying the isolated extremity with blood, from a region of the patient's body that resides outside said isolated extremity, e.g. from the inguinal region.

A major advantage of the present invention over known methods to isolate an extremity, such as the one described by Mumme, is the possibility to perform treatment, e.g. thrombolysis, without having to open a surgical wound, but rather to perform treatment with interventional radiology technique (Seldinger technique) where the vessels are accessed from the inguinal area.

An other advantage of the present invention is that all vessels in an extremity can be treated, not only the ones having a luminal diameter great enough for lodging a catheter as compared to e.g. PCT Application WO 01/70325, where balloons are used to block the blood flow. The present invention is also advantageous over a system with one proximal venous balloon and one proximal arterial balloon in that such a system would not prevent the therapeutic agent from entering the rest of the circulatory system. This because of collateral flow in other vessels than the ones with blocking balloons. Other advantages includes that the apparatus makes treatment possible without the need for general anesthesia, ease of degasifying the perfusion circuit including catheters, possibility to rise the isolated extremity and get rid of contaminated fluid without disconnecting any part of the circuit, possibility to inject contrast solution, and conduct intervention with mechanical means during treatment.

Preferably, the aforementioned fluid circulation loop includes an oxygenation arrangement for oxygenating a fluid passing therethrough, thereby substituting for the long function, a fluid reservoir for providing the capability of volume buffering and bubble trapping, a filter for removing debris such as partly dissolved clots, a shunting arrangement for shunting fluid from an arterial portion of the loop directly to a venous portion of the loop, thereby facilitating priming and degasification of the fluid circulation loop and the catheters, and a heater for adding caloric energy to control the temperature of the circulating fluid.

### DESCRIPTION OF THE DRAWINGS

FIG. 1 shows a system overview of an apparatus according to an embodiment of the invention.

FIG. 2 shows an overview of and an extremity being treated using an apparatus according to an embodiment of the invention.

FIG. 3 shows a reinforced catheter for introduction into a patient's extremity, in accordance with an embodiment of the invention.

FIG. 4 shows a control unit for controlling parameters such as temperature, oxygen saturation and fluid pressure in the isolated extremity, in accordance with an embodiment of the invention.

FIG. 5 shows an arrangement of balloon catheters for treatment of a kidney in a patient.

FIG. 6 shows a cross sectional view of a balloon catheter.

### DESCRIPTION OF THE PREFERRED EMBODIMENTS

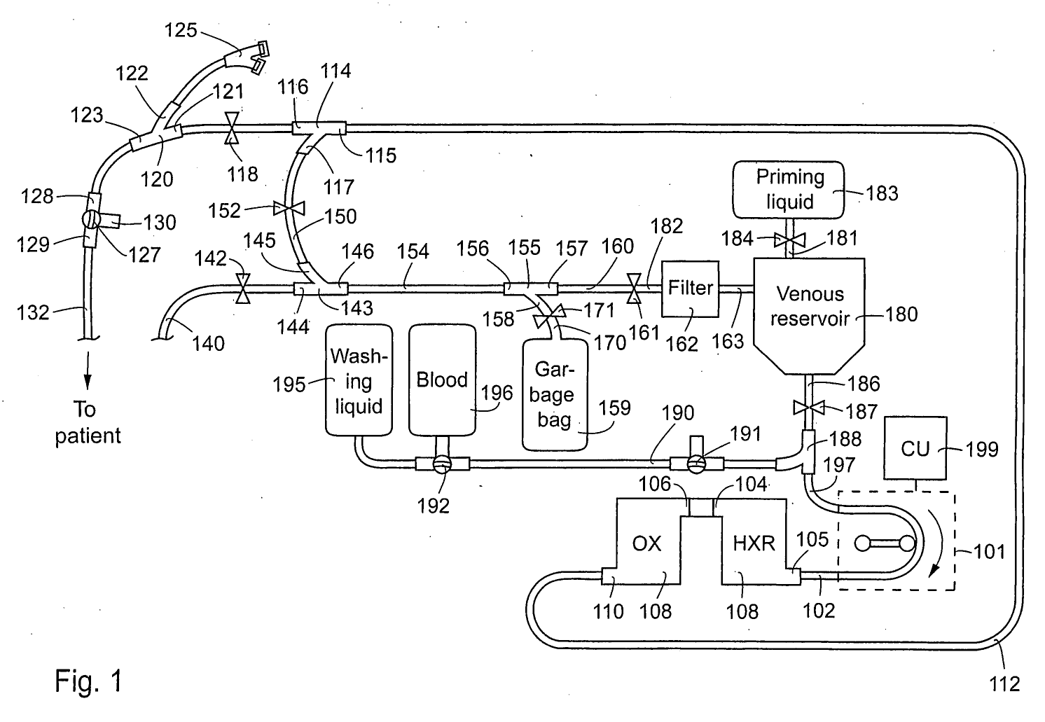

FIG. 1 shows schematically an embodiment of a apparatus according to the invention.

A pump unit 101 is aspirating liquid, which can be venous blood or an artificial solution, from a venous reservoir. Further, the pump 101 is connected via a first liquid transporting conduit 102 to an inlet 105 of a heat-providing unit 103. The heat-providing unit 103 is at its outlet 104 connected to an inlet 106 of an oxygenator unit 108. An outlet 110 of said oxygenator unit 108 is connected via a second liquid transporting conduit 112 to an inlet 115 of a first arterial Y-connector 114.

The first arterial Y-connector 114 is provided with a shunting outlet 117 and a mainstream outlet 116.

The mainstream outlet 116 of the first arterial Y-connector is connected, via a conduit with an arterial pinch clamp 118, to a second arterial Y-connector 120 having a mainstream inlet 121, an auxiliary inlet 122 and a mainstream outlet 123.

The auxiliary inlet 122 is connected to an interventive Y-connector 125 for making it possible to introduce instruments in the patient.

The mainstream outlet 123 of the second arterial Y-connector is connected to a first port 128 of an arterial three-way valve 127 that is suitable for giving contrast injections via a second port 130. A third port of the arterial three-way valve is connected via an arterial trunk conduit 132 to the patient's arterial catheter 202.

On the venous side, blood or liquid coming from the patient via a venous catheter 201 passes a venous front conduit 140. The venous front conduit 140 is provided with a venous pinch clamp 142 and connected to a mainstream inlet 144 of a venous Y-connector 143. A shunt inlet 145 of the venous Y-connector is connected to the shunting outlet 117 and the first arterial Y-connector 114 via a shunt conduit 150 having a shunt pinch clamp 152. The venous Y-connector 143 is also provided with a mainstream outlet 146, which is connected to a first port 156 of a supplementary Y-connector 155 via an intermediate venous conduit 154.

A second port 157 of the supplementary Y-connector 155 is connected to a venous reservoir 180 with an integrated filter 162 via a filter conduit 160 with a filter pinch clamp 161. A third port 158 of the supplementary Y-connector 155 is connected to a collecting bag 159 via a collecting conduit 170 with a collecting pinch clamp 171.

After passing though the integrated filter 162 the liquid is collected in the venous reservoir 180, said venous reservoir has a prime port 181 for connecting a priming liquid bag 183 via a priming conduit having a priming pinch clamp 184.

The venous reservoir 180 is provided with an outlet 186 connected to a first port of a central Y-connector 188, via a conduit provided with a venous outlet pinch clamp 187. A second port of the central Y-connector is connected via a supply conduit 190 with a supply three-way valve 191 to a first port of a liquid selection three-way valve 192. A second and a third port of said liquid selection three-way valve is connected to a washing liquid supply 195 and a blood supply 196.

A third part of the central three-way valve is connected to an inlet 197 of the pump unit 101. The pump is controlled by the control unit 199.

A preferred embodiment of the method according to the invention includes preparing a patient for the treatment by e.g. administering to him or her local anesthetics. Components for the performance of the treatment include a catheter set, a tube set, a pump, a heat exchanger, an oxygenator, a venous reservoir with an integrated filter and some liquids as described above and will be further explained below. In short the following steps are included: priming and degasifying the tube and pumps, catheterization of the patient, degasifying of the catheters, oxygenating and circulating the extremity during the therapeutic act, conducting the therapeutic act, replacing of the therapeutic liquid with blood or first with a washing solution and then with blood, stopping the pump and decatheterization. Below these steps are described in greater detail.

Priming of the Tube and Pump

Priming the tube and pump includes the following steps:
Closing the arterial and venous pinch clamps 118 and 142, opening of the shunt pinch clamp 152 for the shunt 150.
Closing the venous reservoir outlet pinch clamp 187.
Filling the venous reservoir with a suitable liquid e.g. Ringers solution+Mannitol+blood+some tissue recovery agents
Opening the venous reservoir outlet pinch clamp 187 and the central three-way valve in the venous reservoir outlet branch 186 to the air, enabling air to pass out from the tube system.
Closing the three-way valve 191 when air free liquid is pouring out
Closing the pinch clamp 171 to the collecting bag 159.
Starting the pump 101 in order to evacuate air from the oxygenator 108, heat providing unit 103 and the tube set.

Let the pump 101 run for a while. The system is now primed.

Catheterization

Catheterization includes the following steps:
Preparing the patient with e.g. local anesthetics.
Catheterization of the inguinal artery and the inguinal vein in the extremity that is to be treated. (The catheters are described in a separate section below.
The arterial catheter is connected to the arterial front tube 132 and the venous catheter is connected to the venous front tube 140.
Applying a tourniquet for achieving a bloodless field. The distal end of the tourniquet should be applied approximate of the distal end of the catheters.

Degasifying of the Catheters

The degasification process for the catheters includes the following steps:
Opening the arterial pinch clamp 118 and letting air be transported into the venous reservoir 180, this will take just a short moment.
Closing the arterial pinch clamp 118.
Opening the venous pinch clamp for a short moment and letting air pass to the venous reservoir 180.

Circulation and Oxygenation

Circulation and oxygenation include the following steps:
Shutting the pump 101 off.
Closing the shunt pinch clamp 152 on the shunt conduit.
Opening the arterial pinch clamp 118.
Slowly starting the pump 101.
Opening the venous pinch clamp 142.
Increasing the flow and regulating the flow until acceptable pressure has been achieved and the constant level in the venous reservoir 180 is 10 achieved (as small systemic in-flow can be tolerated, but not the opposite). The step of regulating the flow and pressure can be accomplished by the use of a level sensor, sensing the level in the venous reservoir. The sensor is arranged to signal to decrease pump speed if the level in the reservoir decreases. Suitable sensor types include, photocell, pressure sensor, ultrasound, and capacitive sensors, capable of measuring the height of the liquid in the reservoir 180.

The Therapeutic Act

The therapeutic act includes the steps of
Adding to the liquid in the venous reservoir 180 an amount of a therapeutic agent. The agent being different depending on the condition that is treated. For thrombolysis a thrombolytic agent, such as streptokinase, Actilyse® or the like, is suitable. For cancer treatment chemotherapeutic agents are suitable.
Letting the pump 101 work for a couple of minutes.
Optionally, checking the result of the thrombolysis by injecting a contrast liquid in the second port 130 of the arterial three-way valve 127, and performing angiography.

Terminating the Treatment

The termination of the treatment comprises the following steps:
Stopping the pump 101.
Shutting off the pinch clamp 187 between the venous reservoir 180 and the arterial pump 101.
Opening the three-way valve 191 in the supply conduit 190 to the selected washing fluid. Different types of washing fluid include colloid solutions, blood, Ringer's solution. Colored additives such as Evans blue can be added for easier deciding when extremity is washed from thrombolytic agent.
Shutting the incoming venous tube 160 to the venous reservoir 180 between the reservoir inlet and the Y-connector 155.
Opening the pinch clamp 171 to the collecting bag 159.
Starting the pump 101.
Keep the pump 101 running until washing fluid is coming from the venous front conduit.
Stopping the pump 101.
Connecting the liquid solution three-way valve 192 to the blood bag 196.
Starting the pump 101 again.
Keep the pump 101 running until blood is coming from the venous front conduit 190.
Let or roll down a bit of the tourniquet until the valve for letting the pressure out of it is seen.
Release pressure from tourniquet.
Remove tourniquet.
Decatheterization, (reversed catheterization).
Compressing the skin at the insertion sites, to avoid post-treatment bleedings.

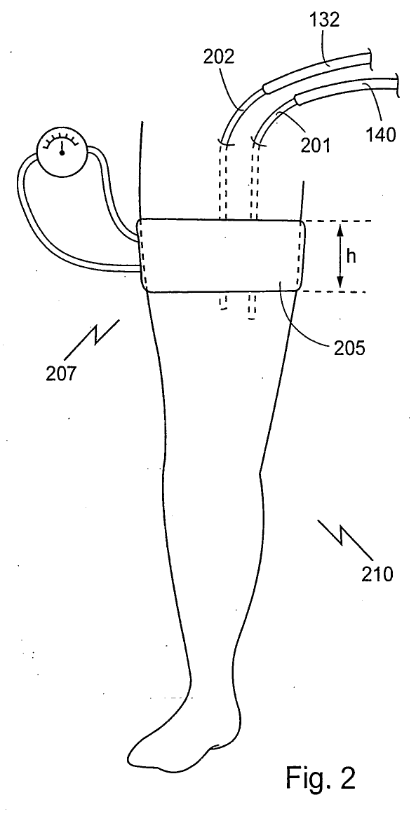

FIG. 2 shows an overview of an extremity that is in the process of being treated using a apparatus according to an embodiment of the invention. The arterial front tube 132 is connected to the arterial catheter 202 that is applied into the femoral artery. The venous front tube 140 is connected to the venous catheter 201 that is applied in the femoral vein. A pressure means in the form of a tourniquet 205 with a height h is applied over a proximal portion 207 of the patient's extremity.

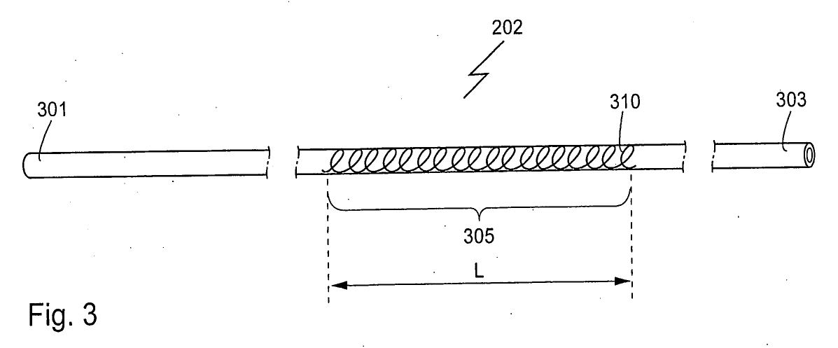

FIG. 3 shows a detailed view of the arterial catheter 202, the venous catheter having the same principal appearance. The catheter 202 has a first end 301, a second end 303 and a reinforced section 305 with a length L. Said reinforced section 305 is devised to make the catheter withstand an outside pressure, i.e giving it structural strength so that it will not collapse when exposed to the pressure of the pressure means 205. The reinforcement can be a metal or composite wire or coil 310 embedded in the catheter wall. The second end 303 has means for being able to connect to the arterial front tube

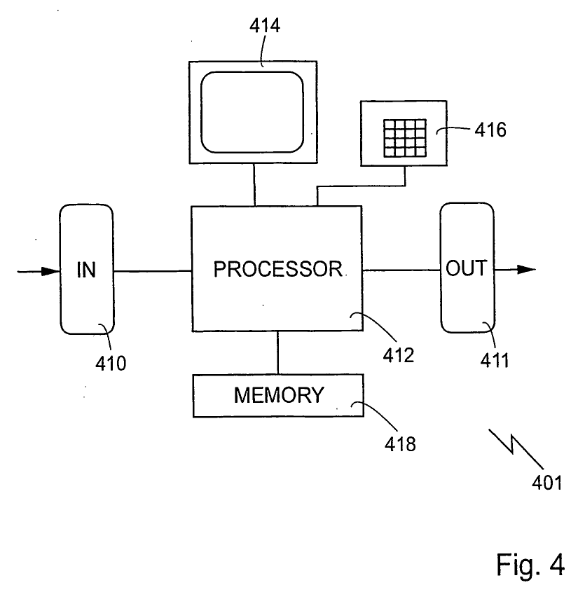

FIG. 4 shows a control unit 401 for controlling parameters such as temperature, oxygen saturation and fluid pressure in the isolated extremity. The control unit comprises an input unit 410 for receiving signals from sensors sensing system parameters in the system such as levels, temperatures, pressures and speeds. The input unit 410 is connected to a processor 412. Said processor 412 processes said parameters and shows them on a display 414. Operator input means is provided via a keyboard 416. A memory unit 418 is provided for storing computer program instructions for instructing the processor 412 and for storing control parameters input from the keyboard 416. The control unit also comprises an output unit for distributing control signals to the pump 101, and in alternative embodiments also to the heat-providing unit 103 and the oxygenator 108 and electronically maneuvered valves replacing some of the pinch clamps.

Another embodiment of the invention comprises a fluid circulation loop connected to balloon catheters forming an efficient apparatus for treating body organs having single or a few arterial and venous connections to the rest of the circulation, such as e.g. the kidneys, the spleen, the liver, the pancreas.

The balloon catheters are provided with means for inflating and deflating the balloon, and are also provided with means for letting a fluid pass through the balloon in such a way that a circulation system can be achieved that circulates therapeutic fluid through the body organ in question, and that therapeutic fluid is prevented from leaking out to the rest of the body circulation.

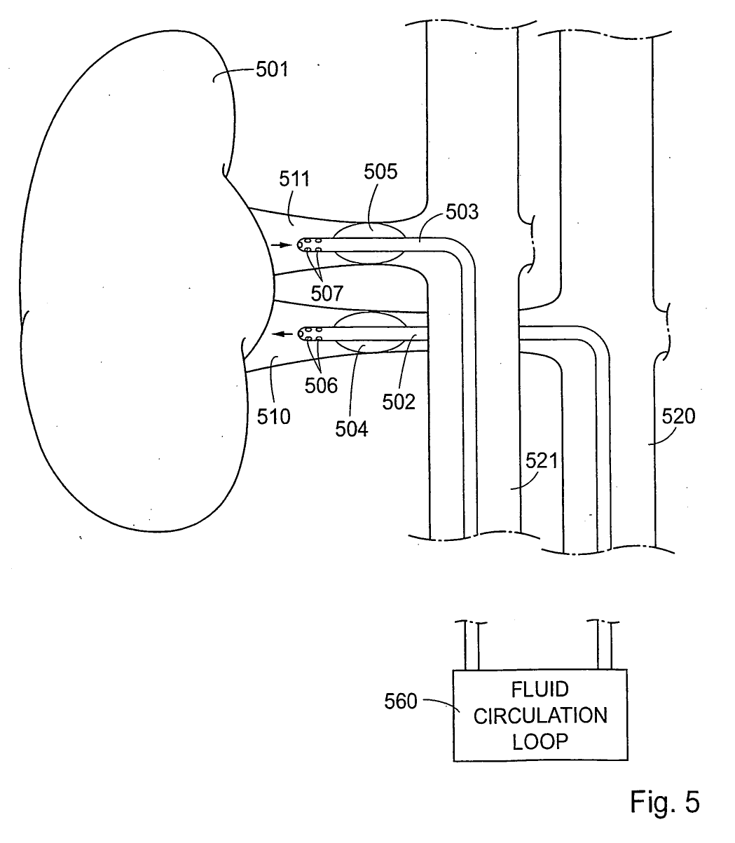

FIG. 5 shows a preferred embodiment useable for treating local cancer in a kidney 501. The embodiment comprises two balloon catheters 502, 503 and a fluid circulation loop 560 in fluid connection with said catheters 502, 503 as described above. In accordance with the invention, said fluid circulation loop 560 is devised as illustrated in FIG. 1 and as further described above in relation thereto.

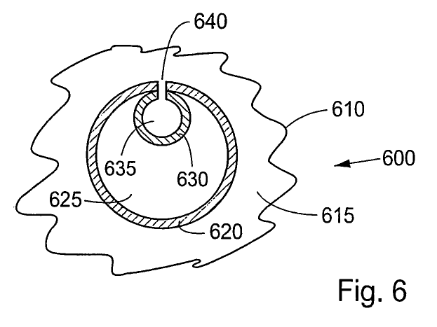

A method for treating cancer using such an embodiment of the invention comprises the steps of introducing a first balloon catheter 502 in the kidney artery 510 of the kidney 501 to be treated, and a second balloon catheter 503 in the kidney vein 511 of the kidney 501 to be treated. Right kidney 501 is shown. The catheters 502, 503 are connected to the fluid circulation loop 560 and the balloons 504, 505 are inflated with a suitable liquid via a separate small diameter tubing e.g. running inside a catheter main lumen 525, see FIG. 6, thereby sealing the kidney artery 510 and vein 511 and shutting off the normal perfusion of the kidney 501 via the aorta 520 and the vena cave inferior 521.

The fluid circulation loop 560 is then activated and is able to perfuse the kidney 501 by introducing oxygenated perfusion fluid via holes 506 in the first catheter 502. Correspondingly, perfusion fluid is drained from the kidney 501 via holes 507 in the second balloon catheter 503.

Cancer treatment with a chemotherapeutic agent or the like is performed by introducing said agent in the perfusion fluid, and circulate said fluid through the kidney.

FIG. 6 shows a cross section of a balloon catheter 600 in the area of the balloon 504, 610. The catheter is provided with a balloon envelope 610, an outer wall 620 and an inner wall 630, which inner wall surrounds an inner lumen 615 via a passage 640. When increasing the pressure in the inner lumen 635 fluid passes through the passage 640 and fills the balloon lumen 615. The diameter of an inflated balloon is arranged to effectively make contact to the walls of a blood vessel providing an efficient obstruction of a blood flow. The balloon envelope 610 is preferably manufactured in a flexible material such as latex eliminating the need for a set of catheters with different diameter balloons, which otherwise would be the case if the balloon is manufactured in a stiff material such as polyurethane.

Although modifications and changes may be suggested by those skilled in the art, it is the invention of the inventors to embody within the patent warranted heron all changes and modifications as reasonably and properly come within the scope of their contribution to the art.

We claim as our invention:

1. An apparatus for circulatory isolation and treatment of a part of a patient's body, said apparatus comprising:
a fluid circulation loop having a first end and a second end;
a flow obstructer adapted for obstructing a blood flow in a patient's blood vessel such that a body part, extremity or organ becomes circulatorily isolated from the circulation of the rest of the body, said flow obstructer including a compressing arrangement, applicable around said extremity to compress a section of said extremity and thereby circulatorily isolate the patient's extremity and prevent a leakage of a fluid from said extremity to the rest of the patient's circulation; and catheters, having a reinforced section, adapted for being introduced in an artery and in a vein having connection to said extremity;
a flow-through arrangement comprising a first flow-through member and a second flow-through member connectable to said first and second ends of said fluid circulation loop, respectively, for providing said part of the patient's body with a fluid connection to the fluid circulation loop, making it possible to circulate a therapeutic fluid through said body part.

2. The apparatus as claimed in claim 1, wherein said compressing arrangement is tourniquet-shaped and has a predetermined height; and said catheters being an arterial catheter having an elongated section that is reinforced to withstand an outside pressure and where said section have a length that is greater than the height of the compressing arrangement; and a venous catheter having an elongated section that is reinforced to withstand an outside pressure, said section having a length that is greater than the height of the compressing arrangement.

3. The apparatus as claimed in claim 1, where said flow obstructer comprises balloon catheters, said catheters being capable of obstructing the blood flow in a patient's blood vessels leading to and from an isolated part or organ of the body, such that said part or organ becomes circulatory isolated from the rest of the body.

4. The apparatus as claimed in claim 1, wherein said apparatus further comprises pump for circulating a fluid in said fluid circulating loop and said catheters.

5. The apparatus as claimed in claim 4, wherein said therapeutic fluid has a composition that allows said fluid to oxygenate and wherein said apparatus comprises an oxygenator unit capable of oxygenating blood and said therapeutic fluid for supplying said extremity with oxygen during a prolonged period of time.

6. The apparatus as claimed in claim 5, wherein said apparatus further comprises a heat providing unit connected in the fluid circulation loop, for warming the fluid.

7. The apparatus as claimed in claim 6, wherein said apparatus comprises a control unit with an input unit for a number of input signal lines and a output unit for a number of control lines devised for controlling a number of process parameters including fluid pressure and temperature, oxygen saturation, and the flow speed of said fluid.

8. The apparatus as claimed in claim 7, wherein said circulatory unit comprises a venous reservoir with an inlet for receiving fluid coming from the patient, and an outlet connected to the pump means for recycling the fluid to the patient.

9. The apparatus as claimed in claim 8, further comprising a level sensor for measuring the height of fluid in the venous reservoir.

10. The apparatus as claimed in claim 9, wherein said control unit comprises a display devised for displaying trend curves and the level of fluid in the venous reservoir and time of treatment.

11. The apparatus as claimed in claim 1 wherein said fluid circulation loop includes a tube set which comprises a shunt connection between an arterial and a venous part of the tube set, said shunt connection having a valve enabling closing and opening of said shunt connection.

12. A method for the treatment of thrombosis in a patient's extremity comprising the steps of:
connecting the internal blood circulatory system of said extremity to an extracorporeal circulation;
isolating the extremity from the rest of the patient's circulation while maintaining an extracorporeal circulation in said extremity; and
adding a thrombolytic agent.

13. A method for the treatment of cancer in a patient's organ comprising the following steps:
connecting the internal blood circulatory system of said organ to an extracorporeal circulation;
isolating the organ from the rest of the patient's circulation while maintaining an extracorporeal circulation of a fluid in said organ;
adding a chemotherapeutic agent to the fluid; and
circulating said fluid with the agent through the organ for achieving a therapeutic effect.

14. The method as claimed in claim 12, further comprising:
catheterizing the patient using Seldinger technique

15. The method as claimed in claim 14 comprising using said Seldinger technique for positioning reinforced catheters in blood vessels passing through tissue compressed from the outside of the patients body.

16. The method as claimed in claim 15, further comprising adding a therapeutic fluid to a fluid in said extracorporeal circulation, said fluid being blood or a therapeutic fluid or a mixture thereof.

17. The method as claimed in claim 16, further comprising oxygenating the fluid.

18. The method as claimed in claim 17, further comprising warming the fluid.

19. The method as claimed in claim 18, where said thrombolytic agent is effective or present in such high concentration that it could cause hemorrhage or other side effect injuries if said liquid entered the blood circulation of the brain or other sensitive organ.

20. An apparatus for treatment of a patient's extremity, said apparatus comprising:
a tourniquet-shaped compressing arrangement having a predetermined height;
an arterial catheter having an elongated section that is reinforced to withstand an outside pressure, said section having a length that is greater than the height of the compressing arrangement;
an venous catheter having an elongated section that is reinforced to withstand an outside pressure and where said section of said venous catheter having a length that is greater than the height of the compressing means.

21. A fluid circulation loop, having a tube connectable to a patient's blood vessel system and useable for altering certain properties of a fluid circulating in said patient, having a first end and a second end connectable to a patient, and a shunt connection.

22. The fluid circulation loop as claimed in claim 21, wherein said shunt connection is connected between a part of the loop which is close to the first end and a part of the loop which is close to the second end of the loop, and in that said shunt connection comprises a valve that enables closing and opening of said shunt connection.

23. The fluid circulation loop as claimed in claim 22, comprising an arterial valve connected in the loop further closer to the first end than the shunt connection, and a venous valve means connected in the loop further closer to the second end than the shunt connection enabling opening and closing of fluid connection to the patient's body.

24. The fluid circulation loop as claimed in claim 23, comprising a fluid removal arrangement for removal of fluid from the loop by a Y-connector and associated valves.

25. The fluid circulation loop as claimed in claim 24, comprising a fluid additive arrangement for adding fluid to the loop by a Y-connector.

26. The fluid circulation loop as claimed in claim 25, comprising a pump, a heater and an oxygenator in said loop.

27. The fluid circulation loop as claimed in claim 24 comprising a valve adapted to add fluid to a venous reservoir when opened.

28. The method as claimed in claim 13, further comprising:
catheterizing the patient using Seldinger technique

29. The method as claimed in claim 28 comprising using said Seldinger technique for positioning reinforced catheters in blood vessels passing through tissue compressed from the outside of the patients body.

30. The method as claimed in claim 29, further comprising adding a therapeutic fluid to a fluid in said extracorporeal circulation, said fluid being blood or a therapeutic fluid or a mixture thereof.

31. The method as claimed in claim 30, further comprising oxygenating the fluid.

32. The method as claimed in claim 31, further comprising warming the fluid.

33. The method as claimed in claim 18, where said thrombolytic agent is effective or present in such high concentration that it could cause hemorrhage or other side effect injuries if said liquid entered the blood circulation of the brain or other sensitive organ.
